(19) United States
(12) Patent Application Publication
(10) Pub. No.: US 2022/0054282 A1
(43) Pub. Date: Feb. 24, 2022

(54) LINEAR ACTUATOR ASSEMBLY, BIONIC DIGIT AND PROSTHETIC HAND

(71) Applicant: EPIC INVENTING INC, Los Angeles, CA (US)

(72) Inventor: Mark HUNTER, Los Angeles, CA (US)

(21) Appl. No.: 17/415,346

(22) PCT Filed: Dec. 19, 2019
(86) PCT No.: PCT/IB2019/061114

§ 371 Date: Jun. 17, 2021

Related U.S. Application Data
(60) Provisional application No. 62/783,739, filed on Dec. 21, 2018.

(51) Int. Cl. A61F 2/58; A61F 2/68

(52) U.S. Cl. CPC A61F 2/586; A61F 2002/701; A61F 2/68

(57) ABSTRACT

A linear actuator assembly housed within a bionic digit, which comprises a base portion, an intermediate portion and an end portion; the intermediate portion pivotally connected to the base portion at one end and pivotally connected to the end portion at the opposite end. The linear actuator assembly includes a drive mechanism; a carriage mechanism having a longitudinal axis (L); a transmission member for transmitting rotational force from the drive mechanism to the carriage mechanism; and a drive member coupled to the carriage mechanism. The carriage mechanism converts the rotational force into an axial force applied to the drive member, moving the drive member along the axis as the carriage mechanism rotates. The drive member is pivotally connected to the end portion. The intermediate portion houses the linear actuator, which further includes a first thrust bearing between the carriage mechanism and the drive mechanism, and the second thrust bearing between the carriage mechanism and a seat provided within the intermediate portion, the carriage mechanism confined between the thrust bearings at each end.

The present invention relates generally to bionic digits, particularly but not exclusively bionic fingers, linear actuator assemblies for bionic digits and carriage assemblies for the linear actuator assemblies, as well as to prosthetic hands including the bionic digits.

Prosthetic hands having one or more movable bionic digit are well known. For example, WO2015138968 discloses a bionic digit comprising a knuckle, a proximal element, a distal element, a force actuator and a rod. The force generator includes a motor that turns a screw and a threaded nut that is coupled to the screw that can be forced to move forward or backward along the axis of the screw as the screw is driven to rotate by the motor. These parts are connected to each other by four pivotal connectors: a first pivotal connector connects the proximal element to the knuckle; the second pivotal connector connects a proximal end of the rod to the knuckle, the second and first connectors being spaced apart; the third pivotal connector connecting the threaded nut to both the distal and the proximal elements, and to the distal end of the rod; and the fourth pivotal connector connecting the distal element to the proximal element, for allowing the distal element to pivot relative to the proximal element, the third and fourth connectors being spaced apart. As the threaded nut is driven along the screw axis, it acts at the third connector to force the distal element to pivot relative to the proximal element at the fourth connector. The rod ensures that the threaded nut remains at a predetermined distance from the second connector, causing the proximal element to rotate relative to the knuckle as the threaded nut is driven to move along the screw axis.

In the above arrangement, the bearings which support the screw assembly are simple roller bearing type lead screw and resists radial movement but is unable to resist axial movement of the lead screw. A circlip is provided on the leadscrew specifically engaging with a surrounding casing to prevent or limit axial movement of the leadscrew. Unfortunately, this circlip simply acts to transfer axial load by means of frictional loading onto the casing and, as such, does not lend itself to efficient operation. Also, in the above arrangement, the leadscrew is bonded to the shaft of the actuator so as to rotate with the shaft as the shaft is rotated. This direct bonding has the undesirable effect of passing any shock loading that the leadscrew might experience due to pressures being exerted thereon by use directly to the actuator itself. Any axial loading of the actuator is undesirable as it may compromise the components within the actuator itself and prevent effective and accurate movements from being achieved.

The present invention is distinct from the above-mentioned prior art in at least two aspects. Firstly, the leadscrew is free to move axially relative to the actuator shaft which prevents any axial loading experienced by the leadscrew from being passed to the actuator and, secondly, the leadscrew is housed between thrust bearings which are mounted in or in association with the casing surrounding the leadscrew. This arrangement allows any axial load experienced by the leadscrew to be directly transferred to the casing but in a manner that reduces friction to a minimum and, thus, provides a more acceptable performance characteristic with less wear and possible damage than is known in the prior art.

Motorised bionic fingers and other parts generate a level of noise when the movement of components is electromechanically actuated, and many known bionic parts are considered to generate undesirable levels of noise. In addition, many known bionic fingers do not exhibit as much grip strength as desired, and it can be particularly challenging to transmit a satisfactory amount of mechanical power to the end segment of a bionic finger. There is a need for bionic digits (including fingers, thumbs and toes) that generate less noise and greater strength when in use. These challenges are addressed by the present invention.

According to a first aspect, there is provided a bionic digit comprising a base portion, an intermediate portion and an end portion. The intermediate portion has a longitudinal axis, proximal end connected to the base portion, and a distal end. The bionic digit has a central plane that includes the longitudinal axis of the intermediate portion. The end portion has a proximal end connected to the distal end of the intermediate portion, and a distal end. A first connector connects the proximal end of the intermediate portion to the base portion, including a pivotal connection for allowing the intermediate portion to pivot about a first pivot axis perpendicular to the central plane. A second connector connects the proximal end of the end portion to the distal end of the intermediate portion, including a pivotal connection for allowing the end portion to pivot relative to the intermediate portion, about a second pivot axis perpendicular to the central plane. The bionic digit may further include a linear actuator assembly, including a drive mechanism for generating a rotational force, having a proximal end and a distal end; a carriage mechanism having a longitudinal axis (which may be parallel to the longitudinal axis of the intermediate portion); a transmission member for transmitting the rotational force from the drive mechanism to the carriage mechanism, the carriage mechanism being interconnected with the transmission member; and a drive member coupled to the carriage mechanism. The carriage mechanism converts the rotational force into an axial force applied to the drive member, moving the drive member along the longitudinal axis as the carriage mechanism rotates, the carriage mechanism having a proximal end and a distal end. A third connector may connect the drive member to the proximal end of the end portion, and may include a pivotal connection between the drive member and the end portion, for allowing the end portion to pivot about a third pivot axis perpendicular to the central plane, relative to the drive member. The intermediate portion may include a housing volume, in which the linear actuator may be housed. The linear actuator assembly may further include a first thrust bearing and a second thrust bearing. The first thrust bearing may be disposed between the proximal end of the carriage mechanism and the distal end of the drive mechanism, axially separating the carriage mechanism from the drive mechanism. The housing volume may include a seat for the second thrust bearing, the seat being at the distal end of the intermediate portion. The second thrust bearing may be disposed between the distal end of the carriage mechanism and the seat, the carriage mechanism being axially confined between the first and second thrust bearings.

According to a second aspect, there is provided a bionic digit comprising a base portion, an intermediate portion and an end portion. The intermediate portion has a longitudinal axis, proximal end connected to the base portion, and a distal end. The bionic digit has a central plane that includes the longitudinal axis of the intermediate portion. The end portion has a proximal end connected to the distal end of the intermediate portion, and a distal end. A first connector connects the proximal end of the intermediate portion to the base portion, including a pivotal connection for allowing the intermediate portion to pivot about a first pivot axis perpendicular to the central plane. A second connector connects the proximal end of the end portion to the distal end of the intermediate portion, including a pivotal connection for allowing the end portion to pivot relative to the intermediate portion, about a second pivot axis perpendicular to the central plane. The bionic digit may further include a linear actuator assembly, including a drive mechanism for generating a rotational force, having a proximal end and a distal end; a carriage mechanism having a longitudinal axis (which may be parallel to the longitudinal axis of the intermediate portion); a transmission member for transmitting the rotational force from the drive mechanism to the carriage mechanism, the carriage mechanism being interconnected with the transmission member; and a drive member coupled to the carriage mechanism. The carriage mechanism converts the rotational force into an axial force applied to the drive member, moving the drive member along the longitudinal axis as the carriage mechanism rotates, the carriage mechanism having a proximal end and a distal end. A third connector may connect the drive member to the proximal end of the end portion, and may include a pivotal connection between the drive member and the end portion, for allowing the end portion to pivot about a third pivot axis perpendicular to the central plane, relative to the drive member. The intermediate portion may include a housing volume, in which the linear actuator may be housed. The transmission member may be interconnected with the carriage mechanism, for allowing the drive mechanism to drive rotation of the carriage mechanism, while allowing the carriage mechanism to move axially along the transmission member, within the limits asserted by the first and second thrust bearings. The interconnection being a slidable interconnection where the carriage mechanism is free to slide over the surface of the drive mechanism, the axial constraint of such movement being limited only by the first and second thrust bearings.

Viewed from a further aspect, there is provided linear actuator assembly for a bionic digit that includes a housing for the linear actuator, the linear actuator comprising: a drive mechanism for generating a rotational force, having a proximal end and a distal end; a carriage mechanism having a longitudinal axis; a transmission member for transmitting the rotational force from the drive mechanism to the carriage mechanism, the carriage mechanism being interconnected with the transmission member; and a drive member coupled to the carriage mechanism. The carriage mechanism converts the rotational force into an axial force applied to the drive member, moving the drive member along the longitudinal axis of the carriage mechanism as the carriage mechanism rotates; the carriage mechanism having a proximal end and a distal end. The linear actuator further comprises a first thrust bearing and a second thrust bearing. The first thrust bearing disposed between the proximal end of the carriage mechanism and the distal end of the drive mechanism, axially separating the carriage mechanism from the drive mechanism. The second thrust bearing can be disposed between the distal end of the carriage mechanism and the distal end of the intermediate portion, the carriage mechanism being axially confined between the first and second thrust bearings. A screw assembly can be provided for the linear actuator assembly, the screw assembly comprising the carriage mechanism (for example, a threaded rod), a drive member (for example, a matingly threaded nut), and the first and second thrust bearings.

Viewed from a further aspect, there is provided a prosthetic hand comprising a disclosed bionic digit.

Disclosed linear actuator assemblies, as well as bionic digits and prosthetic hands that comprise them may exhibit an unexpectedly large decrease in the level of noise generated by the linear actuator in use. This is likely to be highly desirable for users wearing prosthetic hands and/or bionic fingers. In addition, example arrangements may exhibit a high level of efficiency, i.e., low levels of energy dissipation in use, and the strength of the grip of example bionic fingers may be substantially increased. Furthermore, the speed of pivoting the intermediate portion and the end portion of the bionic digit may be substantially increased.

The present disclosure envisages various example arrangements of bionic digits and linear actuator assembles for bionic digits, including various optional features and combinations of features, non-limiting and non-exhaustive examples of which are briefly described below.

The transmission member is interconnected with the carriage mechanism for driving the rotation of the carriage mechanism. In some example arrangements, the interconnection may allow the carriage mechanism to move axially along the transmission member substantially freely, as axially constrained by the first thrust bearing and second thrust bearings. In other words, the carriage mechanism may be able to move substantially freely along the transmission member, to the extent that this may be permitted by the first and second thrust bearings. Such an arrangement is elsewhere herein described as “axially separating” the carriage mechanism from the drive mechanism. In such arrangements, the transmission member cannot transmit substantial axial forces between the carriage mechanism and the drive mechanism. In other example arrangements, the carriage mechanism may be adhered to the transmission member by means of adhesive material, or the interconnection of the carriage mechanism and the transmission member may limit, or substantially prevent, axial movement of the carriage mechanism relative to the transmission member.

The central plane of the bionic digit may include the longitudinal axis of the carriage mechanism, which may be parallel and/or coincident with the longitudinal axis of the intermediate portion. Opposite sides of the digit, on either side of the central plane, may be referred to a left- and right-hand sides of the digit (viewed from the base portion towards the end portion). In some example arrangements, a bionic finger may be substantially symmetric about an axis and/or a plane; however, this disclosure is not limited to reflectively symmetric bionic digits. Although connectors and certain other features are generally referred to herein in the singular, it would be straightforward for the skilled person to envisage these being present in pairs, in which a pair of connectors consists of respective connectors for the left-hand side and the right-hand side of the digit; each pair of pivotal connectors allowing pivoting about a common respective pivot axis, which passes through both of the pair of pivotal connections. Connectors and certain other features may be present as pairs of left- and right-hand connectors regardless of whether or not the digit is symmetric about a central plane.

In some example arrangements, the drive mechanism may include a drive motor for generating torque and a planetary gear system for converting a torque generated by the drive motor to a torque applied to the transmission member. The drive motor and the gear system may be contained within the same housing, or in different housings.

The drive member may be coupled to the intermediate portion by a fourth connector, including a translational connection between the drive member and the intermediate portion, for allowing the drive member to move translationally along the axis of the carriage mechanism relative to the intermediate portion, whilst preventing the drive member from rotating about the axis, relative to the intermediate portion. As used herein, a ‘translational connection’ between two bodies indicates that the connection allows the two bodies to move a distance in a straight line relative to each other, in direct or indirect contact with each other, at the connection. For example, a translational connection may allow the connected bodies to slide against each other, although other arrangements of translational connections are encompassed. As an example of a translational connection, a pin may extend from one of the connected bodies and other of the connected bodies may include a slot receiving the pin, so that the bodies can slide relative to each other of a distance determined by the length of the slot. So, in other words, a fourth connector connecting the drive member and the intermediate portion may allow the drive member to move over a limited distance along the intermediate portion, whilst preventing the drive member from rotating about the longitudinal axis of the carriage mechanism, relative to the intermediate portion when the carriage mechanism rotates in use.

In an example arrangement of the fourth connector, the intermediate portion may have one or more side walls including a slot, which receives a pin, rod or disc (for example) projecting laterally from the drive member and extending through the slot. The slot may be parallel to the axis of the carriage mechanism along which the drive member moves, the opposite ends of the slot limiting the distance of axial travel of the drive member. Corresponding slots may be provided on opposite left- and right-hand sides of the intermediate portion, each receiving a respective projection from the drive member.

The carriage mechanism can convert a torque applied to it by the drive mechanism to an axial force applied to the drive member coupled to it, the axial force urging the drive member to move translationally in either direction along the axis of the carriage mechanism. For example, the carriage mechanism may comprise a threaded rod and the drive member may comprise a body that includes a threaded portion for mating with the threaded rod (as a nut can be coupled to a screw or bolt, for example). As the threaded rod is driven to rotate about its longitudinal axis (which may be considered as a ‘screw axis’), the threading converts the rotation into an axial force acting on the drive member, forcing it to move along the axis in a direction determined by the direction of the rotation (clockwise or anti-clockwise).

In some example arrangements, the carriage mechanism may not be directly attached to the drive mechanism and torque generated by the drive mechanism is transmitted to the carriage mechanism by the transmission member, which may comprise or consist of a drive shaft. A proximal end of the drive shaft may be attached to the drive mechanism for receiving torque generated by the drive mechanism, and project along the longitudinal axis of the carriage mechanism (and along which axis the drive member is to travel), a distal end of the drive shaft being remote from the drive mechanism. The carriage mechanism may include a recess into its proximal end, for receiving the drive shaft coaxially. The threaded rod may not be axially attached to the drive shaft and, at least when not fully assembled with the other parts, the threaded rod can slide substantially freely (except for unavoidable friction) along the drive shaft. However, the threaded rod may be mechanically coupled to the drive shaft to cause the carriage mechanism to rotate about its axis when the drive shaft is being driven to rotate, whilst the mechanical coupling may not constrain axial movement between the threaded rod and the drive shaft. For example, the cross-section of the drive shaft and the recess may be polygonal (e.g., hexagonal), although many other mechanical interconnection arrangements are envisaged. Alternatives include simple splined arrangements and arrangements with keyways installed and a key between the two components in question.

In examples where the carriage mechanism is not attached directly to the transmission member, it may be possible to substantially reduce or avoid small axial movements of the carriage mechanism from resulting in a significant axial force applied to the drive mechanism, particularly to a gear system. In other example arrangements, the threaded rod may be axially attached to the drive shaft.

In some example arrangements, the intermediate portion may comprise an encasement having an internal volume, within which the linear actuator is wholly or partly housed. The internal volume may be elongate, having proximal and distal ends corresponding to those of the intermediate portion as a whole. At least one of the ends of the internal volume may be open. For example, the proximal end may be open, or capable of being opened to insert or remove the linear actuator. The encasement may include a fastener for attaching at least the drive mechanism, or a housing of a drive mechanism, to the encasement. For example, the internal volume may include a threaded region, and a region of the exterior surface of the drive mechanism may include mating threading, for allowing the linear actuator to be fastened to the intermediate portion by threaded interconnection. Other ways of fastening the drive mechanism or other parts of the linear actuator to the intermediate portion are also envisaged. Thus, the generation of torque by the drive mechanism will not cause the intermediate portion to rotate relative to the linear actuator.

An example thrust bearing system may comprise a pair of opposing race rings, and a plurality of rolling bearing units housed within a bearing cage between the race rings, for allowing the opposing race rings to rotate relative to each other about a common axis though the centres of both race rings, with little or substantially negligible loss of energy to friction (that is, a thrust bearing may be considered to be highly efficient). Example bearing units may include ball bearings, or cylindrical or conical roller bearings. In various example arrangements, the first and second thrust bearings may be of the same type, and/or the same size; or different types of thrust bearing systems may be used. Some types of ball thrust bearings may be viewed as having reflective symmetry about a plane midway between the opposing race rings, while certain other types of thrust bearings may not have reflective symmetry about a centre plane. In the latter case, it may be more preferable to arrange the thrust bearing in a particular orientation with respect to the adjacent components.

The first thrust bearing system is positioned at a proximal end of the carriage mechanism, and comprises first and second opposing race rings. The first race ring (or a housing that contains it) may abut the distal end of the drive mechanism, and/or a portion of the encasement of the intermediate portion. The opposing second race ring is more remote from the drive mechanism and should be as free as possible to rotate relative to the first race ring. The transmission member may extend from the drive mechanism, coaxially through the centre of the race rings of the first thrust bearing system, and the proximal end of the carriage mechanism may abut, or be attached to, the second race ring. In some example arrangements, the first thrust bearing may contact the carriage mechanism and the drive mechanism on opposite sides, or the first thrust bearing may be spaced apart from the drive mechanism.

The encasement of the intermediate portion includes a seat for receiving the second thrust bearing, or a device comprising the second thrust bearing. The seat may comprise a portion of the internal volume surface, being shaped for receiving a first of the race rings of the thrust bearing, or for seating a housing within which the second thrust bearing is mounted. Whilst the first race ring is held within, or abuts, the seat, the opposing second race ring may rotate substantially freely against the bearing elements, which roll between the race rings along race paths. A distal end of the carriage mechanism may be attached to, or abut, the second race ring. The second thrust bearing may thus contact the distal end of the carriage mechanism on one side, and seat on the opposite side, whilst substantially reducing the dissipation of mechanical energy of the rotating carriage mechanism onto the intermediate portion.

The disclosed example arrangements of the carriage mechanism confined between first and second thrust bearings allows the carriage member to rotate efficiently as it is driven by the transmission member. The arrangement may significantly reduce, or minimise, transmission of axial force between the carriage mechanism and the drive mechanism via the transmission member, and enhances the rotational efficiency of the carriage mechanism.

Some example bionic digits may comprise one or more support elements having a proximal end and a distal end (for example, one support element on either side of the digit). A proximal end region of the support element may be connected to the base portion by a fifth connector, and a distal end region of the support element may be connected to the drive member by a sixth connector. The fifth connector may include a pivotal connection, for allowing the support element to pivot relative to the base portion, and the sixth connector may include a pivotal connection for allowing the support element to pivot relative to the drive member. For example, the distal end region of the support element may include an aperture for receiving a pin extending from the drive member.

In example arrangements that include support elements, the support element limits the distance between the drive member and the fifth connector (and the base portion). The fifth connector may be spaced apart from the first connector on the base portion, and consequently, the support element and the intermediate portion may pivot about different pivot axes and describe non-concentric arcs when they pivot. Since the support element constrains the distance between the drive member and the fifth connector (and the base portion), when the drive member is forced by the drive mechanism to move along the axis of the carriage mechanism, the intermediate portion can be forced to pivot about the base portion (at the first connector) in the plane including the axis of the carriage mechanism. For example, when the drive member is positioned fully forward, toward the distal end of the carriage mechanism, the finger may be as fully outstretched as possible, and as the drive member is pulled backwards, towards the distal end of the carriage mechanism, it may pull the lower region of the end portion inward, causing the end portion to pivot at the second connector relative to the intermediate portion; simultaneously, the support element may push the intermediate portion downwards, maintaining the distance between the drive member and the fifth connector.

Other elements of the present invention are detailed in the appended claims.

Non-limiting example arrangements of screw mechanisms, bionic digits and prosthetic hands will be described with reference to the accompanying drawings, of which:

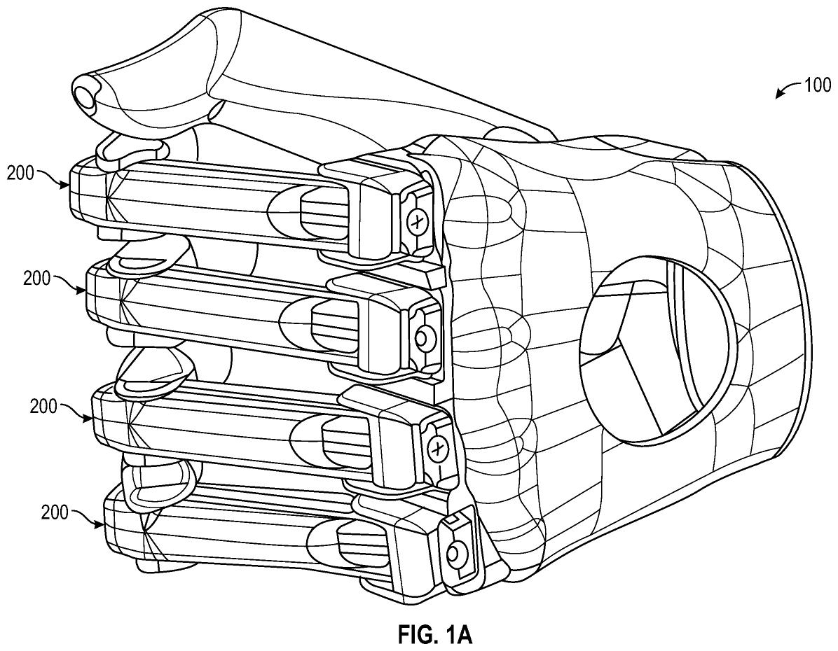

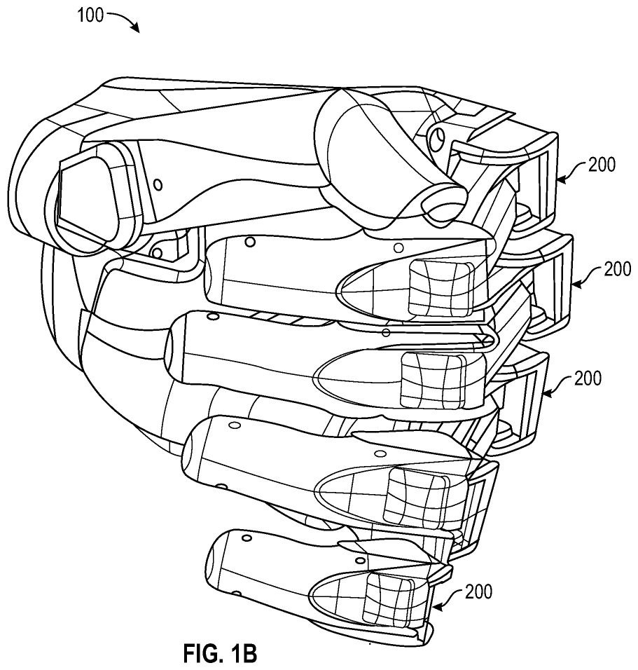

FIG. 1A shows a schematic top view of an example of a partly closed prosthetic hand, showing top views of the knuckles and the intermediate portions; and

FIG. 1B shows a schematic underside view of the example prosthetic hand, showing the tops of the end portions of example bionic fingers;

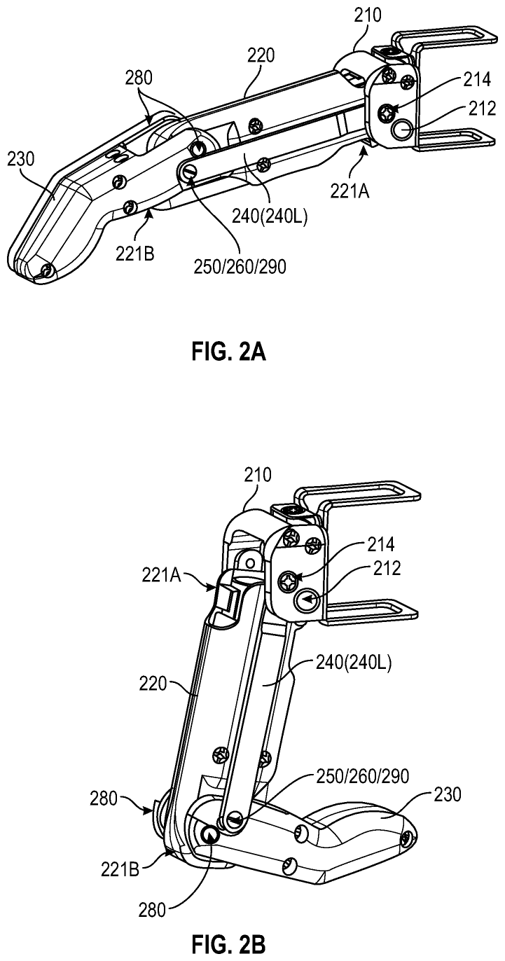

FIG. 2A shows a schematic perspective view of an example bionic finger in the fully open, extended position (although the third 260, fourth 250 and sixth 290 connectors are indicated in this drawing because they share a common pivot pin that extends from the drive member, only the sixth 290 is visible); and

FIG. 2B shows a schematic perspective view of the bionic finger in the fully closed, curled-inward position;

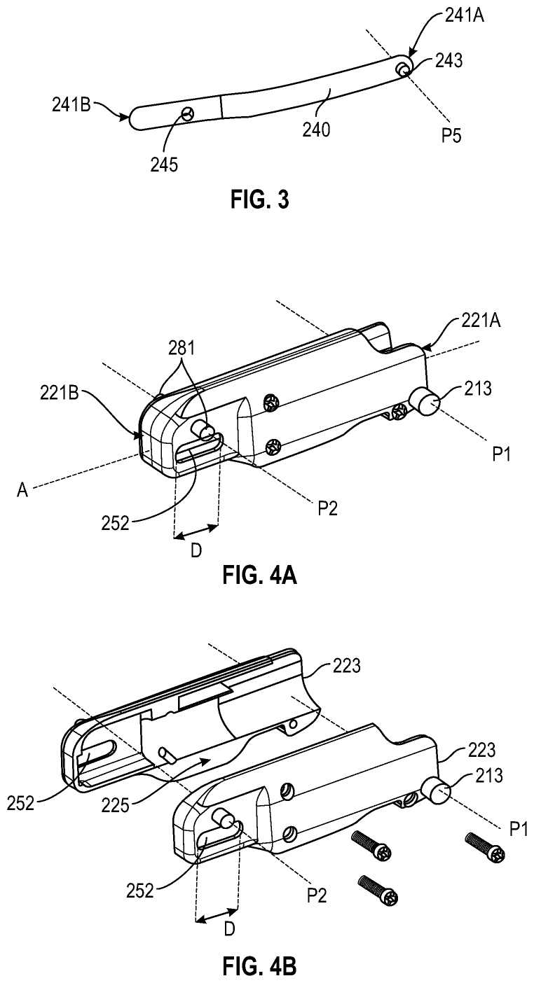

FIG. 3 shows a schematic perspective view of an example support element for a bionic finger;

FIG. 4A shows a schematic perspective view of an example intermediate portion, showing its longitudinal axis A; and

FIG. 4B shows a schematic exploded perspective view of the example intermediate portion;

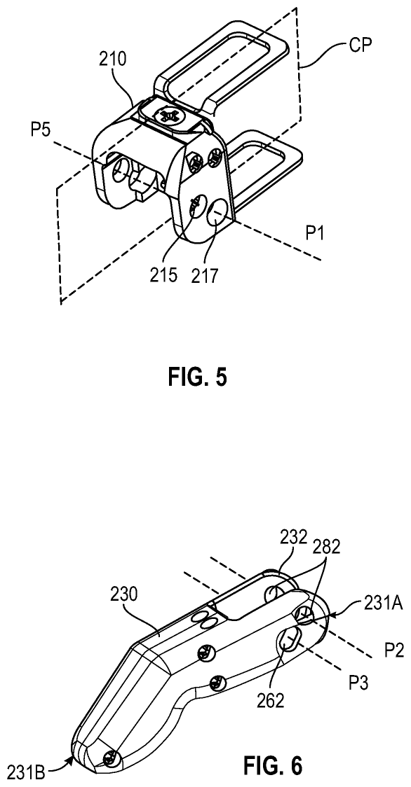

FIG. 5 shows a schematic perspective view of an example base portion for a bionic finger, including the central plane CP;

FIG. 6 shows a schematic perspective view of an example end portion for a bionic finger;

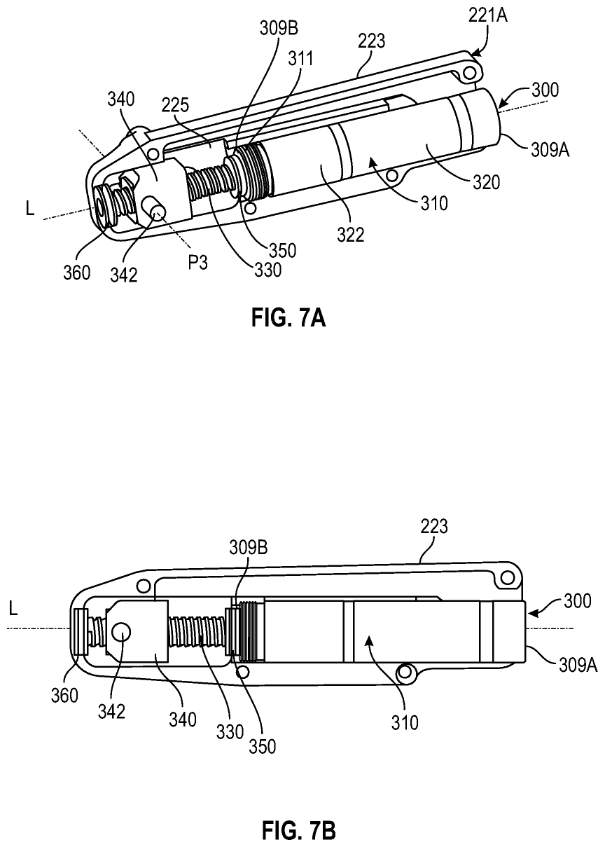

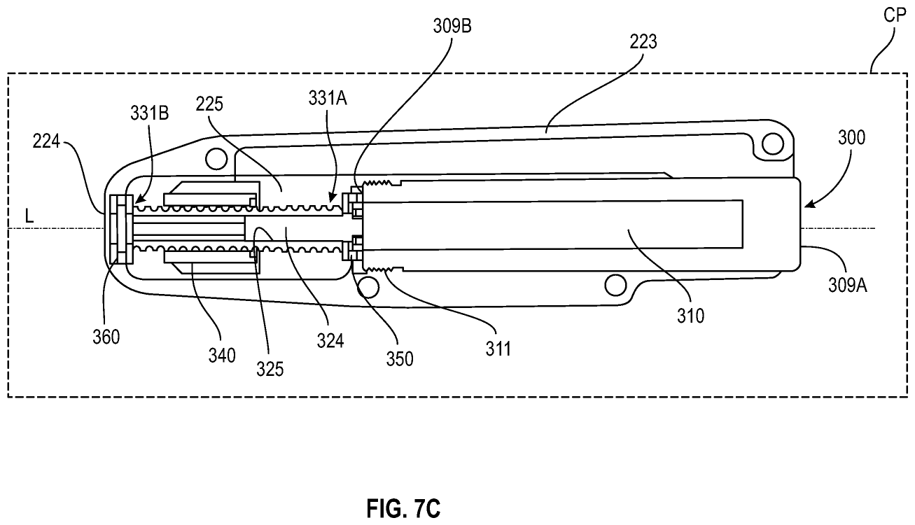

FIG. 7A shows a schematic cut-away side perspective view of an example intermediate portion, showing the linear actuator assembly, in which the drive motor is fastened to an encasement of the intermediate portion;

FIG. 7B shows a schematic cut-away side view of the example intermediate portion, showing the linear actuator assembly; and

FIG. 7C shows a schematic view of a central longitudinal cross-section through the intermediate portion, showing the linear actuator assembly, showing the central plane CP;

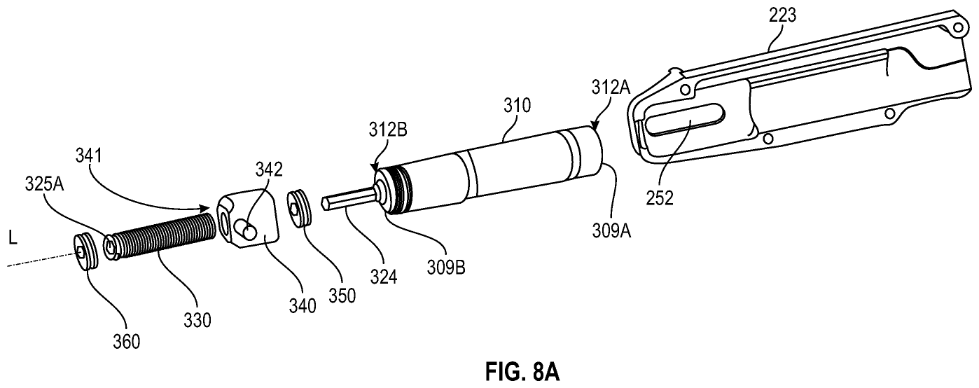

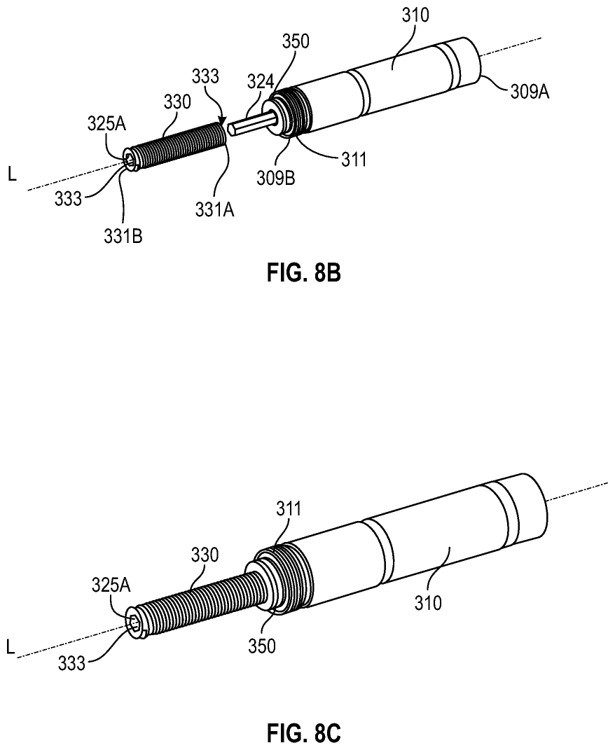

FIG. 8A shows a schematic exploded perspective view of an example linear actuator, with part of the encasement of the intermediate portion;

FIG. 8B shows a schematic exploded perspective view of part of the example linear actuator, showing a drive shaft projecting from the drive mechanism, and a threaded rod carriage mechanism removed from the drive shaft; and

FIG. 8C shows a schematic perspective view of part of the example linear actuator, in which the threaded rod carriage mechanism is mounted onto the drive shaft extending from the drive mechanism;

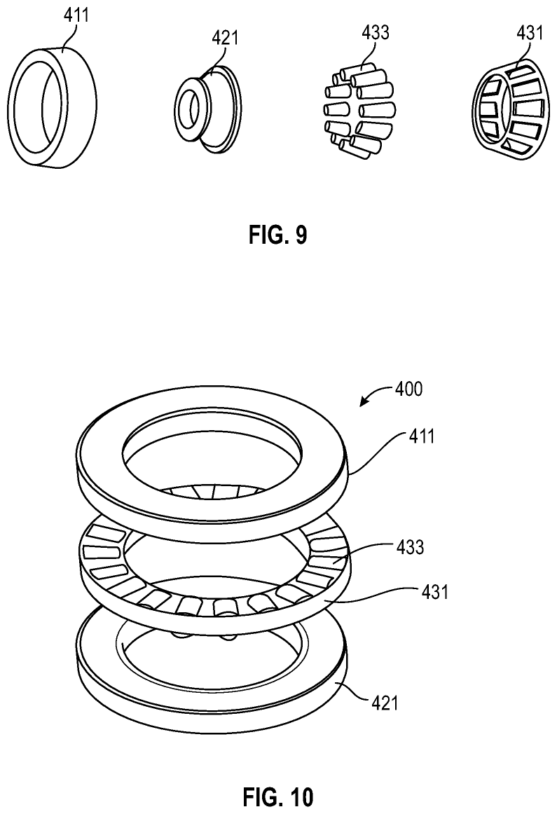

FIG. 9 shows a schematic exploded perspective view of a conical roller thrust bearing;

FIG. 10 shows a schematic partly exploded perspective view of an example of a cylindrical roller thrust bearing;

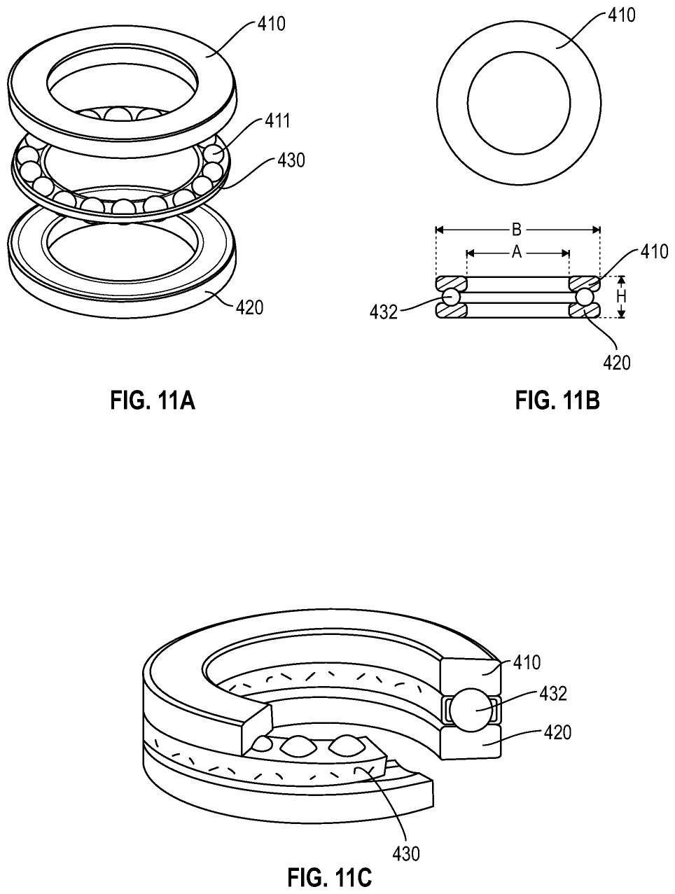

FIG. 11A shows a schematic partly exploded perspective view of an example of a ball thrust bearing;

FIG. 11B shows a schematic top view and a cross-section view through the ball thrust bearing;

FIG. 11C shows a schematic partly cut-away perspective view of an example ball thrust bearing;

FIG. 12 is a general view of the actuator assembly;

FIG. 13 is an exploded view of the drive member and thrust bearing arrangements;

FIG. 14 is an assembled view of the drive member and thrust bearing arrangements;

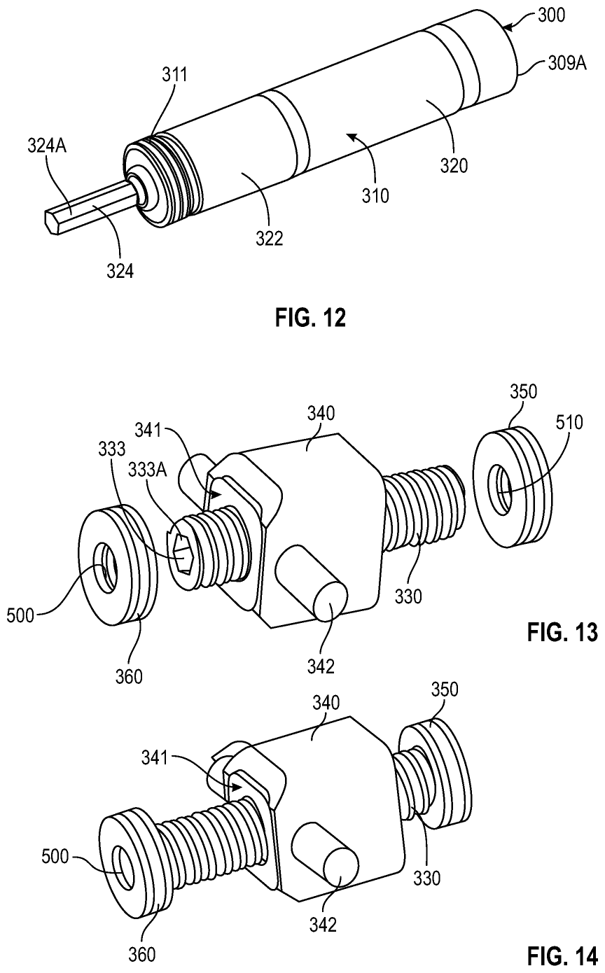

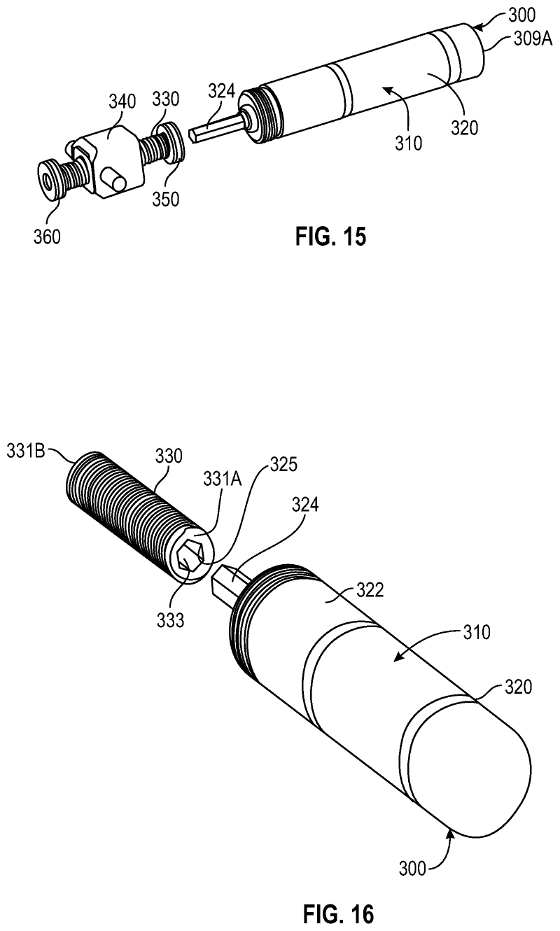

FIG. 15 is an exploded view of the drive member and thrust bearings shown in-line with the actuator with which they become coupled for rotational movement therewith; and

FIG. 16 is a view of the actuator and the carriage from a different angle such as to show the arrangement for the drive shaft

With reference to FIGS. 1A and 1B, an example prosthetic hand 100 comprises five example bionic fingers 200 and a bionic thumb. The prosthetic hand 100 can be fitted to a user by means of an attachment sleeve (not shown);

With reference to FIGS. 2A to 8C, an example bionic finger 200 comprises: a base portion 210, which may be considered as corresponding to a knuckle; an intermediate portion 220; an end portion 230; a linear actuator assembly 300 (shown in more detail in FIGS. 7A-7C) and a pair of support arms 240, on the right- and left-hand side of the digit, of which an example is shown in FIG. 3. As used herein, a linear actuator is a device that generates force to drive a body in a straight line. The intermediate portion 220 has a proximal end 221A connected to the base portion 210 and a distal end 221B, which is axially displaced from the proximal end 221A (as used herein unless otherwise indicated, the ‘end’ of a member or element includes an end region that is coterminous with the end). The end portion 230 is pivotally connected to the distal end 221B of the intermediate portion 220. The intermediate portion 220 has a longitudinal axis A and the bionic finger 200 has a central plane CP that includes the longitudinal axis A, defining left- and right-hand sides of the bionic digit 200, on either side of the central plane CP (viewed from the base portion 210 towards the end portion 230). The bionic digit 200 may be substantially symmetric (particularly but not exclusively having reflective symmetry) about the central plane CP.

In the illustrated example arrangement, the intermediate portion 220 comprises an encasement 223 defining a housing volume 225 within which the linear actuator 300 is housed. The encasement 223 comprises two longitudinal halves (a left-hand half and a right-hand half), as illustrated in FIG. 4B. In this example, the housing volume 225 is elongate, being open at the proximal end 221A of the intermediate portion 220 and closed at the distal end 221B.

An example linear actuator assembly 300 is illustrated in detail in FIGS. 7A-7C and comprises a drive mechanism 310, a threaded rod 330 (a specific example of a carriage mechanism), a drive shaft 324 (a specific example of a transmission member), a threaded nut 340 (a specific example of a drive member), and first and second thrust bearings 350, 360. The drive mechanism 310 comprises a motor 320 for generating torque, and a planetary gear system 322, and has a proximal end 309A and a distal end 309B. The threaded rod 330 has a proximal end 331A and a distal end 331B, an elongate recess 333 provided into the proximal end 331A for receiving the drive shaft 324. The recess 333 is coaxial with the longitudinal axis L of the threaded rod 330, and the drive shaft 324 transmits torque from the drive mechanism 310 to the threaded rod 330. The drive shaft 324 extends from the drive mechanism 310 and is inserted into the recess 333 of the threaded rod 330. The gear system 322 converts a torque generated by the motor 320 to a rotational force applied to the drive shaft 324, and consequently to the threaded rod 330. The drive mechanism 310 is coupled in a fixed relationship to the intermediate portion 220, so that that the drive mechanism 310 cannot rotate relative to the intermediate portion 220 in use. The exterior of the drive mechanism 310 includes a threaded region 311 that mates with a corresponding threaded region of the encasement 223, within the housing volume 225, so that the drive mechanism 310 is threadedly interconnected with the encasement 223.

In this example, the cross-sections of the recess 333 and the drive shaft 324 are both hexagonal in shape, the mating hexagonal shapes providing azimuthal inter-connection between the drive shaft 324 and the threaded rod 330 via contact surfaces 325A and 325B respectively, enabling the drive shaft 324 to turn the threaded rod 330. In this example, there is no adhesive between the threaded rod 330 and the drive shaft 324, and the drive shaft 324 has uniform cross-sectional dimensions and shape along its length, thus allowing the threaded rod 330 to move substantially freely along the drive shaft 324 within the constraints of the first and second thrust bearings 350, 360. In other words, the threaded rod 330 is mechanically mounted onto the drive shaft 324, interconnected with the drive shaft 324 in a way that the threaded rod 330 is prevented from rotating relative to the drive shaft 324, but is not prevented by the drive shaft 324 from sliding axially on the drive shaft 324 along the longitudinal axis L of the threaded rod 330. Consequently, the drive shaft 324 cannot transmit an axial force between the drive mechanism 310 and the threaded rod 330. In other examples, different mechanical configurations and polygonal shapes may be used for rotationally interconnecting the drive shaft 324 and the threaded rod 330. The longitudinal axis L of the threaded rod 330 is parallel to the longitudinal axis A of the intermediate portion 220 in this example (and may be so in general). The threaded nut 340 includes a threaded through-hole 341, in which the threading of the threaded nut 340 mates with the threading of the threaded rod 330, and the threaded nut 340 is threadedly coupled to the threaded rod 330.

The bionic finger includes at least three pairs of connectors, each pair consisting of corresponding connectors on either side of the finger (i.e., on the left- and right-hand side of its central axis):
a) a first connector 212 pivotally connects the proximal end 221A of the intermediate portion 220 to the base portion 210,
b) a second connector 280 pivotally connects the proximal end 231A of the end portion 230 to the distal end 221B of the intermediate portion 220, and
c) a third connector 260 pivotally connects the threaded nut 340 to the end portion 230.

Each of the left-hand and right-hand first connectors 212 comprises a respective pivot pin 213 defining a first pivot axis P1 (coaxial with both of the first pivot pins 213) and extending from the proximal end 221A of the intermediate portion 220 into corresponding apertures 217 on the left- and right-hand sides of the base portion 210. The pivotal connections 213 of the first connectors 212 allow the intermediate portion 220 to pivot about the first pivot axis P1 relative to the base portion 210, within the central plane CP (as shown in FIGS. 4A to 5).

Each of the second connectors 280 comprises a respective pivot pin 281 defining a second pivot axis P2 (coaxial with both of the pivot pins 281) and extending from the distal end 221B of the intermediate portion 220 into corresponding apertures 282 in the left- and right-hand sides of the proximal end 231A of the end portion 230. The pivotal connection 281 of the second connector 280 allows the end portion 230 to pivot about the second pivot axis P2, relative to the intermediate portion 220.

The threaded nut 340 threadedly coupled to the threaded rod 330 includes a pair of pivot pins 342 extending from left- and a right-hand sides thereof, defining a third pivot axis P3 (coaxial with both of the first pivot pins 342). Each third connector 260 comprises the respective pivot pin 342 extending from a respective side of the threaded nut 340 into a respective aperture 262 in the then portion 230.

The end portion 230 has a proximal end 231A and a distal end 231B, and includes a pair of flanges 232 coterminous with the proximal end 231A, on either side of the bionic digit 200. Each of the flanges 232 includes a respective first aperture 282 for receiving a respective pivot pin 281 of the intermediate portion 220 (forming the second connector 280), as well as a respective second aperture 262 for receiving the respective pivot pin 342 extending from a respective side of the threaded nut 340 (forming the third connector 260). When the threaded nut 340 moves along the threaded rod 330 in response to the threaded rod being 330 driven to rotate, the threaded nut 340 acts at the third connectors 260 to move the end portion 230, causing the end portion 230 to pivot at the second connector 280 about the second pivot axis P2. Viewing a prosthetic hand 100 as having a lower side and an upper side, in which the fingers can curl away from the upper side and towards a palm of a bionic hand on the lower side, the second connectors 280 are positioned above the third connectors 260.

With reference to FIGS. 2A, 2B, 4A and 4B, the example bionic finger 200 includes a pair of fourth connectors 250, each comprising a respective one of the pivot pins 342 of the threaded nut 340 extending into a respective slot 252 in the encasement 223 of the intermediate portion 220. Each slot 252 extends parallel to the longitudinal axis L of the threaded rod 330. This arrangement of the pivot pins 342 through the slots 252 on either side of the bionic digit 200 allows the threaded nut 340 to move along the longitudinal axis L of the threaded rod 330 between the ends of the slots 252 (the fourth connector 250 is indicated in FIGS. 2A and 2B because it shares the drive member pivot pin 342 with the third connector 260 and the sixth connector 290, of which only the sixth connector 290 is visible in this view). The axial movement of the threaded nut 340 is thus limited by length D of the slots 252, and the slots 252 receiving the pivot pins 342 substantially prevent the threaded nut 340 from rotating relative to the intermediate portion 220, about the axis L of the threaded rod 330.

With reference to FIGS. 7A to 7C, the linear actuator assembly 300 includes a first thrust bearing 350 and a second thrust bearing 360, the threaded rod 330 being axially confined between the first and second thrust bearings 350, 360. The encasement 223 includes a seat 224 for accommodating the second thrust bearing 360 at a distal end of the housing volume 225, adjacent the distal end 221B of the intermediate portion 220. The first thrust bearing 350 is positioned between the proximal end 331A of the threaded rod 330 and the distal end 309B of the drive mechanism 310, and the second thrust bearing 360 is positioned between the distal end 331B of the threaded rod 330 and the seat 224. Both the first and the second thrust bearings 350, 360 may be ball thrust bearing systems 400, examples of which are illustrated in FIGS. 9A-9C, although other types of thrust bearings may be used in other example arrangements.

In some examples, the threaded rod 330 may be axially unbound to the draft shaft 224 and may be capable of moving substantially freely (axially) along the drive shaft 330 to a limited extent that may be permitted by the first and second thrust bearings 350, 360. In other words, the first and second thrust bearings 350, 360 may allow the threaded rod 330 to rotate substantially freely, its rotation being determined by drive shaft 324. In some example arrangements, the axial movement of the threaded rod 330 may be constrained only by the thrust bearings 350, 360 at either end of thereof.

With reference to FIGS. 9-11C, example thrust bearings comprise a first race ring 410, 411 and a second race ring 420, 421, between which ball bearings 432 or roller bearings 433 are held within a cage 430, 431, allowing the first race ring 410, 411 and second race ring 420, 421 to rotate efficiently with respect to one another, with low or negligible frictional energy loss.

A first race ring 411, 410 of the first thrust bearing 350 may be attached to, or abut, the distal end 309B of the drive mechanism 310 in some example arrangements. An opposing second race ring 421, 420 of the first thrust bearing 350 can rotate substantially freely relative to the first race ring 411, 410. The proximal end 331A of the threaded rod 330 may abut the second race ring 421, 420 of the first thrust bearing 350; the proximal end 331A of the threaded rod 330 may or may not be attached to the second race ring 421, 420 of the first thrust bearing 350.

The encasement 223 of the intermediate portion 220 includes a seat 224 at the distal end of the housing volume 225, for seating a first race ring 410, 411 of the second thrust bearing 360. An opposing second race ring 420, 421 of the second thrust bearing 360 can rotate substantially freely relative to the first race ring 410, 411 of the second thrust bearing 360. The distal end 331B of the threaded rod 330 may abut the second race ring 420, 421 of the second thrust bearing 360; the distal end 331B of the threaded rod 330 may or may not be attached to the second race ring 420, 421 of the second thrust bearing 360. The threaded rod 330 is thus confined between the rotatable race rings of each of the first and second thrust bearings 350, 360.

When the threaded rod 330 is driven to rotate about its axis L, the threaded nut 340 is forced to move axially within the limits permitted by the slots 252. The arrangement of the drive mechanism 310, drive shaft 324, threaded rod 330, threaded nut 340 and slots 252 thus converts a torque generated by the drive mechanism 310 into an axial force that drives the threaded nut 340 to move axially, and consequently to exert a force on the end portion 230 at the third connector 260, forcing the end portion 230 to pivot at the second connector 280 relative to the intermediate portion 220.

With reference to FIGS. 2A, 2B and 3, an example bionic digit 200 may include a pair of support arms 240, one on either side of the central plane, each having a respective proximal end 241A and a distal end 241B. Each support arm 240 connects the drive member 340 with the base portion 210, constraining the distance between the drive member 340 and the base portion 210. A fifth connector 214 pivotally connects a proximal end 241A of the support arm 240 with the base portion 210. In this example, each left and right support arm 240 includes a respective pivot projection 243 at the proximal end 241A, for insertion into an aperture 215 in the base portion 210 and defining a pivot axis P5, for allowing the support element 240 to pivot about the pivot axis P5, relative to the base portion 210. A sixth connector 290 pivotally connects a distal end 241B of the support arm 240 with the drive member 340. In this example, the sixth connector 290 includes an aperture 242 at the distal end of the support arm 240, into which the pivot pin 342 of the threaded nut 340 projects. In FIGS. 2A and 2B, the third connector 260, fourth connector 250 and sixth connector 290 are indicated as being coincident because they share the same pivot pin 342 of the drive member 340, although only the sixth connector 290 is visible in this view.

The first connector 212 (pivotal about P1) and the fifth connector 214 (pivotal about P5) are spaced apart from each other on the base portion 210, the fifth connector 214 positioned above the first connector 212 on the base portion 210. The support arms 240 and the intermediate portion 220 thus have different pivot axes about the base portion 210, and consequently, the longitudinal axis L of the threaded rod 330 has a different effective pivot axes about the base portion 210 than the support arms 240.

The support arms 240 limit the distance between the threaded nut 340 and the base portion 210, consequently limiting the arrangement of the bionic digit 200, including the position of the intermediate portion 220 relative to the base portion 210 and the position of the end portion 230 relative to the intermediate portion 220, depending on the position of the threaded nut 340 along the axis L of the threaded rod 330. With reference to FIG. 2A, the bionic digit 200 can be put into a fully extended arrangement, in which the threaded nut 340 is as far towards the distal end 221B of the intermediate portion 220 as is permitted by the slots 252 in the encasement 223. With reference to FIG. 2B, the bionic digit 200 can be put into a fully closed, or curled, arrangement, in which the threaded nut 340 is as far towards the proximal end 221A of the intermediate portion 220 as is permitted by the slots 252. The position of the bionic digit 200 may thus be controlled by the threaded nut 340 being driven to move axially in response to the drive mechanism 310 applying torque to the threaded rod 330 via the drive shaft 324, and as constrained by the support arms 240.

Reference will now be made to FIGS. 12 to 17 which more clearly illustrate the connection between the drive shaft 324 and the threaded rod 330 and also between the threaded rod 330 and the thrust bearings 350, 360. From these figures it will be appreciated that the drive shaft 324 will have an external surface 324A which, as shown, includes one or more keying surfaces thereon, as discussed in detail above. The keying surface 324A is matched with an equal but opposite keying surface 333A within the recess 333 within the threaded rod 330 such that the keying surfaces engage with one another and rotate together upon actuation of the actuator to cause rotational movement of the drive shaft 324. The threaded rod 330 is free to slide axially along the drive shaft 324 within the limits set by the thrust washers 350, 360 provided at the first and second ends 331A, 331B of the threaded rod 330. As a consequence of the above-described arrangement, any axial shock loading experienced by the drive member 340 due to the finger being impacted by an external load is first transferred to the threaded rod 330 upon which it is located but is not then transferred to the drive shaft 324 as the threaded rod 330 will simply slide axially along the driveshaft 324 itself. The axial movement of the threaded rod 330 will be arrested by one or other of the thrust washers 350, 360 disposed at opposite ends of the threaded rod and secured to the casing itself. These thrust bearings are as shown in FIGS. 9 to 11 and, effectively, arrest axial movement whilst allowing low friction rotation to be maintained.

1. A bionic digit comprising:
a base portion; an intermediate portion and an end portion;
the intermediate portion having a longitudinal axis (A), a proximal end connected to the base portion, and a distal end;
the bionic digit having a central plane (CP) that includes the longitudinal axis (A) of the intermediate portion;
the end portion having a proximal end connected to the distal end of the intermediate portion, and a distal end;
a first connector connecting the proximal end of the intermediate portion to the base portion, including a pivotal connection for allowing the intermediate portion to pivot about a first pivot axis (P1) perpendicular to the central plane (CP);
a second connector connecting the proximal end of the end portion to the distal end of the intermediate portion, including a pivotal connection for allowing the end portion to pivot relative to the intermediate portion, about a second pivot axis (P2) perpendicular to the central plane (CP);
a linear actuator assembly, including a drive mechanism for generating a rotational force and having a proximal end and a distal end;
a carriage mechanism having a longitudinal axis (L);
a transmission member interconnected with the carriage mechanism for transmitting the rotational force from the drive mechanism to the carriage mechanism; and
a drive member coupled to the carriage mechanism; the carriage mechanism converting the rotational force into an axial force applied to the drive member, moving the drive member along the longitudinal axis (L) as the carriage mechanism rotates; the carriage mechanism having a proximal end and a distal end;
a third connector connecting the drive member to the proximal end of the end portion, including a pivotal connection between the drive member and the end portion, for allowing the end portion to pivot about a third pivot axis (P3) perpendicular to the central plane (CP), relative to the drive member;
the intermediate portion including a housing volume, housing the linear actuator;
wherein the linear actuator assembly further includes a first thrust bearing and a second thrust bearing;
the transmission member being interconnected with the carriage mechanism, for allowing the drive mechanism to drive rotation of the carriage mechanism, while allowing the carriage mechanism (330) to move axially along the transmission member, as axially constrained by the first and second thrust bearings.

2. A bionic digit comprising:
a base portion; an intermediate portion and an end portion;
the intermediate portion having a longitudinal axis (A), a proximal end connected to the base portion, and a distal end;
the bionic digit having a central plane (CP) that includes the longitudinal axis (A) of the intermediate portion;
the end portion having a proximal end connected to the distal end of the intermediate portion, and a distal end;
a first connector connecting the proximal end of the intermediate portion to the base portion, including a pivotal connection for allowing the intermediate portion to pivot about a first pivot axis (P1) perpendicular to the central plane (CP);
a second connector connecting the proximal end of the end portion to the distal end of the intermediate portion, including a pivotal connection for allowing the end portion to pivot relative to the intermediate portion, about a second pivot axis (P2) perpendicular to the central plane (CP);
a linear actuator assembly, including a drive mechanism (310) for generating a rotational force and having a proximal end and a distal end;
a carriage mechanism (330) having a longitudinal axis (L);
a transmission member interconnected with the carriage mechanism for transmitting the rotational force from the drive mechanism to the carriage mechanism; and
a drive member coupled to the carriage mechanism; the carriage mechanism converting the rotational force into an axial force applied to the drive member, moving the drive member along the longitudinal axis (L) as the carriage mechanism rotates; the carriage mechanism (330) having a proximal end and a distal end;
a third connector connecting the drive member to the proximal end of the end portion, including a pivotal connection between the drive member and the end portion, for allowing the end portion to pivot about a third pivot axis (P3) perpendicular to the central plane (CP), relative to the drive member;
the intermediate portion including a housing volume, housing the linear actuator;
wherein the linear actuator assembly further includes a first thrust bearing and a second thrust bearing;
the first thrust bearing disposed between the proximal end of the carriage mechanism and the distal end of the drive mechanism; and
the housing volume includes a seat at the distal end of the intermediate portion, for seating the second thrust bearing, the second thrust bearing disposed between the distal end of the carriage mechanism and the seat, the carriage mechanism being axially confined between the first and second thrust bearings.

3. The bionic digit as claimed in claim 2, the transmission member being interconnected with the carriage mechanism, for allowing the drive mechanism to drive rotation of the carriage mechanism, while allowing the carriage mechanism to move axially along the transmission member, as axially constrained by the first and second thrust bearings.

4. The bionic digit as claimed in claim 1, the drive mechanism including
a drive motor for generating torque, and
a planetary gear system for converting a torque generated by the drive motor (320) to a torque applied to the transmission member.

5. The A bionic digit as claimed in claim 1, wherein the drive member is coupled to the intermediate portion by a fourth connector;
the fourth connector connecting the drive member and the intermediate portion, for allowing the drive member to move over a distance (D) along the intermediate portion, whilst preventing the drive member from rotating about the longitudinal axis (L) of the carriage mechanism, relative to the intermediate portion when the carriage mechanism rotates in use.

6. The bionic digit as claimed in claim 1, wherein
the carriage mechanism comprises a threaded rod and
the drive member includes a mating screw-threaded portion for interconnecting with the threaded rod, wherein the threading converts rotation of the carriage mechanism to movement of the drive member along the longitudinal axis (L) of the threaded rod.

7. The bionic digit (200) as claimed in claim 1, wherein
the transmission member, and
the carriage mechanism into its proximal end, receiving the drive shaft, the recess being coaxial with the longitudinal axis (L) of the carriage mechanism.

8. The bionic digit as claimed in claim 7, wherein the recess and the drive shaft have the same cross-sectional polygonal shape.

9. The bionic digit as claimed in claim 1, the intermediate portion comprising
an encasement having an interior volume defining the housing volume, wherein the encasement and the drive mechanism have a complementary fastening mechanism between them.

10. The bionic digit as claimed in claim 1, one or both of the first thrust bearing and the second thrust bearing being a thrust ball bearing system, comprising ball bearings, a ball cage for supporting the ball bearings, a first race ring and a second race ring; wherein the ball bearings are supported between the first and second race rings by the ball cage.

11. The bionic digit as claimed in claim 1, at least one of the first thrust bearing and the second thrust bearing being a roller bearing system, comprising roller bearings, a roller cage for supporting the roller bearings, a first race ring and a second race ring; wherein the roller bearings are supported between the first and second race rings by the roller cage.

12. The bionic digit as claimed in claim 1, wherein the first and second thrust bearings are of the same type.

13. The bionic digit as claimed in claim 1, the bionic digit having right- and left-hand sides on laterally opposite sides of the central plane (CP); each side having respective first connectors, second connectors and third connectors; each pair of left- and right-hand connectors having the same respective pivot axis (P1, P2, P3).

14. The bionic digit as claimed in claim 1, comprising a support element having a proximal end connected to the base portion and a distal end connected to the drive member;
a fifth connector connecting the proximal end of the support element to the base portion, including a pivotal connection for allowing the support element to pivot relative to the base portion, about a pivot axis (P5) perpendicular to the central plane (CP); and
a sixth connector connecting the distal end of the support element to the drive member, including the pivotal connection for allowing the support element to pivot relative to the drive member, about the third pivot axis (P3) perpendicular to the central plane (CP).

15. The bionic digit as claimed in claim 14, comprising
a right-hand support element and a left-hand support element,
the right-hand support element connecting the base portion to the drive member on the right-hand side of the bionic digit; and
the left-hand support element connecting the base portion to the drive member on the left-hand side of the bionic digit;
respective fifth connectors on the right-hand side and the left-hand side, connecting the proximal ends of the respective support elements to the respective side of the base portion, and
respective sixth connectors on the right-hand side and the left-hand side, connecting the distal ends of the respective support elements to the respective pivotal connections extending from the respective sides of the drive member.

16. A linear actuator assembly for a bionic digit that includes a housing for accommodating the linear actuator assembly; the linear actuator assembly comprising:
a drive mechanism having a proximal end and a distal end, for generating a rotational force;
a carriage mechanism having a longitudinal axis (L), a proximal end and a distal end;
a transmission member for transmitting the rotational force from the drive mechanism to the carriage mechanism, the carriage mechanism being interconnected with the transmission member; and
a drive member coupled to the carriage mechanism; the carriage mechanism converting the rotational force into an axial force applied to the drive member, moving the drive member along the longitudinal axis (L) as the carriage mechanism rotates;
a first thrust bearing and
a second thrust bearing; wherein
the first thrust bearing is disposed between the proximal end of the carriage mechanism and the distal end of the drive mechanism; and
the second thrust bearing can be disposed between the distal end of the carriage mechanism and a portion of the housing, the carriage mechanism being axially confined between the first and second thrust bearings.

17. The linear actuator assembly as claimed in claim 16, the transmission member being interconnected with the carriage mechanism, for allowing the drive mechanism to drive rotation of the carriage mechanism, while allowing the carriage mechanism to move axially along the transmission member, as axially constrained by the first and second thrust bearings, the transmission member not transmitting axial forces between the carriage mechanism and the drive mechanism.

18. The linear actuator assembly as claimed in claim 16, the drive mechanism including:
a drive motor for generating torque, and
a planetary gear system for converting a torque generated by the drive motor to a torque applied to the transmission member.

19. The linear actuator assembly as claimed in claim 16, wherein
the carriage mechanism comprises a threaded rod and
the drive member includes a mating screw-threaded portion for interconnecting with the threaded rod, wherein the threading converts rotation of the carriage mechanism to movement of the drive member along the longitudinal axis (L) of the threaded rod.

20. The linear actuator assembly as claimed in claim 16, wherein
the transmission member comprises a drive shaft, and
the carriage mechanism includes a recess into its proximal end receiving the drive shaft, the recess being coaxial with the longitudinal axis (L) of the carriage mechanism.

21. The linear actuator assembly as claimed in claim 20, wherein the recess and the drive shaft have the same cross-sectional polygonal shape.

22. The linear actuator assembly as claimed in claim 16, one or both of the first thrust bearing and the second thrust bearing being a thrust ball bearing system, comprising ball bearings, a ball cage for supporting the ball bearings, a first race ring and a second race ring; wherein the ball bearings are supported between the first and second race rings by the ball cage.

23. The linear actuator assembly as claimed in claim 16, at least one of the first thrust bearing and the second thrust bearing being a roller bearing system, comprising roller bearings, a roller cage for supporting the roller bearings, a first race ring and a second race ring; wherein the roller bearings are supported between the first and second race rings by the roller cage.

24. The linear actuator assembly as claimed in claim 16, wherein the first and second thrust bearings are of the same type.

25. The prosthetic hand comprising a bionic digit as claimed in claim 1.
